(19) United States
(12) Patent Application Publication
(10) Pub. No.: US 2025/0354947 A1
(43) Pub. Date: Nov. 20, 2025

(54) METHOD FOR LARGE-AREA SOIL MONITORING BY DISTRIBUTED TEMPERATURE SENSING

(71) Applicant: NEC Laboratories America, Inc., Princeton, NJ (US)

(72) Inventors: Yue TIAN, Princeton, NJ (US); Yangmin DING, East Brunswick, NJ (US); Zhuocheng JIANG, Plainsboro, NJ (US); Sarper OZHARAR, Pennington, NJ (US); Ting WANG, West Windsor, NJ (US)

(73) Assignee: NEC Laboratories America, Inc., Princeton, NJ (US)

(21) Appl. No.: 19/206,519

(22) Filed: May 13, 2025

Related U.S. Application Data
(60) Provisional application No. 63/648,715, filed on May 17, 2024.
(60) Provisional application No. 63/648,743, filed on May 17, 2024.

(51) Int. Cl. G01N 25/56; G01N 33/24

(52) U.S. Cl. CPC G01N 25/56; G01N 33/246

(57) ABSTRACT

Disclosed are systems and methods that employ distributed fiber optic sensing (DFOS)/distributed temperature sensing (DTS) to measure/monitor soil moisture and seepage at sub-meter spatial resolution over a large geographic area. In sharp contrast to the prior art which generally employed many, point soil moisture sensors, systems and methods according to aspects of the present disclosure employ DTS with an fiber optic sensor that advantageously senses soil temperature at sub-meter spatial resolution continuously. From these continuous, wide areas, high resolution DTS measurements, soil moisture and seepage determinations of soil in which the fiber optic sensor contacts are made.

### CROSS REFERENCE TO RELATED APPLICATIONS

This application claims the benefit of U.S. Provisional Patent Application Ser. No. 63/648,715 filed May 17, 2024, and U.S. Provisional Patent Application Ser. No. 63/648,743 filed May 17, 2024, the entire contents of each of which is incorporated by reference as if set forth at length herein.

### FIELD OF THE INVENTION

This application relates generally to distributed fiber optic sensing (DFOS) systems, methods, and structures. More particularly, it pertains to improved DFOS/Distributed Temperature Sensing (DTS) systems and methods that measure soil moisture and water seepage in soil and earthen embankments respectively.

### BACKGROUND OF THE INVENTION

As those skilled in the art will understand and appreciate, the ability to determine and quantify soil moisture levels is critically important to many industries including agriculture, forestry, civil engineering, water resource management, environmental monitoring, and landscaping. However, across a large geographic area, it is difficult to monitor soil moisture levels changes at a fine spatial resolution as traditional point soil moisture sensors only monitor a single point. To monitor a large area at fine spatial resolution, many point soil moisture sensors are needed and may require additional data communication facilities to support data transmission from and among such sensors. Additionally, such point sensors operate using local power supplies or batteries, which are expensive in term of long-term maintenance cost.

### SUMMARY OF THE INVENTION

An advance in the art is made according to aspects of the present disclosure directed to systems and methods that employ distributed fiber optic sensing (DFOS)/distributed temperature sensing (DTS) to measure/monitor soil moisture and seepage at sub-meter spatial resolution over a large geographic area. In sharp contrast to the prior art which generally employed many, point soil moisture sensors, systems and methods according to aspects of the present disclosure employ DTS with an fiber optic sensor that advantageously senses soil temperature at sub-meter spatial resolution continuously. From these continuous, wide areas, high resolution DTS measurements, soil moisture determinations of soil in which the fiber optic sensor contacts are made.

### BRIEF DESCRIPTION OF THE DRAWING

FIG. 1(A) and FIG. 1(B) are schematic diagrams showing an illustrative prior art uncoded and coded DFOS systems.

FIG. 2 is a schematic diagram showing operational details of an illustrative system according to aspects of the present disclosure.

FIG. 3 is a schematic diagram showing an illustrative fiber optic sensor layout over a large geographic area in which DTS measurements are made according to aspects of the present disclosure.

FIG. 4 is a schematic flow diagram showing illustrative steps involved in DTS and soil moisture measurements and determinations according to aspects of the present disclosure.

FIG. 5 is a schematic flow diagram showing illustrative steps involved in DTS soil moisture measure measurements and determinations according to aspects of the present disclosure.

FIG. 6 is a schematic flow diagram showing illustrative steps involved in DTS soil temperature measurement and soil moisture levels and determining a seepage according to aspects of the present invention.

FIG. 7 is a schematic block diagram of an illustrative computer system in which aspects of the present disclosure may be executed.

### DETAILED DESCRIPTION OF THE INVENTION

The following merely illustrates the principles of this disclosure. It will thus be appreciated that those skilled in the art will be able to devise various arrangements which, although not explicitly described or shown herein, embody the principles of the disclosure and are included within its spirit and scope.

Furthermore, all examples and conditional language recited herein are intended to be only for pedagogical purposes to aid the reader in understanding the principles of the disclosure and the concepts contributed by the inventor(s) to furthering the art and are to be construed as being without limitation to such specifically recited examples and conditions.

Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Thus, for example, it will be appreciated by those skilled in the art that any block diagrams herein represent conceptual views of illustrative circuitry embodying the principles of the disclosure.

Unless otherwise explicitly specified herein, the FIGs comprising the drawing are not drawn to scale.

By way of some additional background, we note that distributed fiber optic sensing systems convert the fiber to an array of sensors distributed along the length of the fiber. In effect, the fiber becomes a sensor, while the interrogator generates/injects laser light energy into the fiber and senses/detects events along the fiber length.

As those skilled in the art will understand and appreciate, DFOS technology can be deployed to continuously monitor vehicle movement, human traffic, excavating activity, seismic activity, temperatures, structural integrity, liquid and gas leaks, and many other conditions and activities. It is used around the world to monitor power stations, telecom networks, railways, roads, bridges, international borders, critical infrastructure, terrestrial and subsea power and pipelines, and downhole applications in oil, gas, and enhanced geothermal electricity generation. Advantageously, distributed fiber optic sensing is not constrained by line of sight or remote power access and—depending on system configuration—can be deployed in continuous lengths exceeding 30 miles with sensing/detection at every point along its length. As such, cost per sensing point over great distances typically cannot be matched by competing technologies.

Distributed fiber optic sensing measures changes in “backscattering” of light occurring in an optical sensing fiber when the sensing fiber encounters environmental changes including vibration, strain, or temperature change events. As noted, the sensing fiber serves as sensor over its entire length, delivering real time information on physical/environmental surroundings, and fiber integrity/security. Furthermore, distributed fiber optic sensing data pinpoints a precise location of events and conditions occurring at or near the sensing fiber.

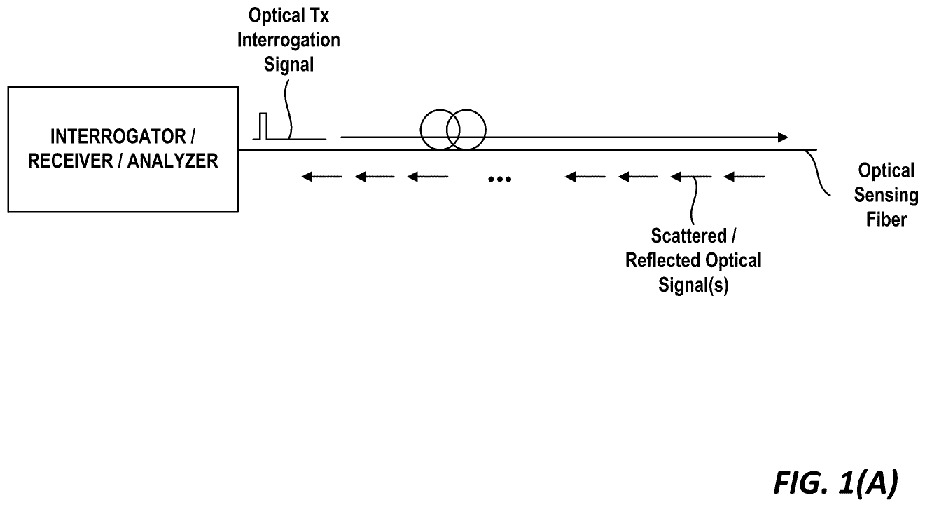

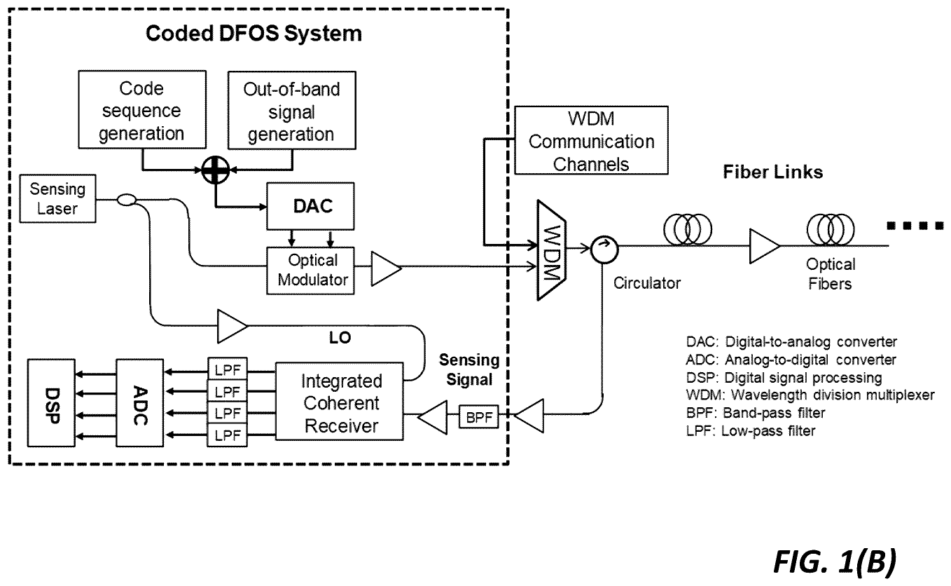

A schematic diagram illustrating the generalized arrangement and operation of a distributed fiber optic sensing system that may advantageously include artificial intelligence/machine learning (AI/ML) analysis is shown illustratively in FIG. 1(A). With reference to FIG. 1(A), one may observe an optical sensing fiber that in turn is connected to an interrogator. While not shown in detail, the interrogator may include a coded DFOS system that may employ a coherent receiver arrangement known in the art such as that illustrated in FIG. 1(B).

As is known, contemporary interrogators are systems that generate an input signal to the optical sensing fiber and detect/analyze reflected/backscattered and subsequently received signal(s). The signals received are analyzed, and an output is generated which is indicative of the environmental conditions encountered along the length of the fiber. The backscattered signal(s) so received may result from reflections in the fiber, such as Raman backscattering, Rayleigh backscattering, and Brillion backscattering.

As will be appreciated, a contemporary DFOS system includes the interrogator that periodically generates optical pulses (or any coded signal) and injects them into an optical sensing fiber. The injected optical pulse signal is conveyed along the length optical fiber.

At locations along the length of the fiber, a small portion of signal is backscattered/reflected and conveyed back to the interrogator wherein it is received. The backscattered/reflected signal carries information the interrogator uses to detect, such as a power level change that indicates—for example—a mechanical vibration or an indication of temperature.

The received backscattered signal is converted to electrical domain and processed inside the interrogator. Based on the pulse injection time and the time the received signal is detected, the interrogator determines at which location along the length of the optical sensing fiber the received signal is returning from, thus able to sense the activity of each location along the length of the optical sensing fiber. Classification methods may be further used to detect and locate events or other environmental conditions including acoustic and/or vibrational and/or thermal along the length of the optical sensing fiber.

Distributed acoustic sensing (DAS) is a technology that uses fiber optic cables as linear acoustic sensors. Unlike traditional point sensors, which measure acoustic vibrations at discrete locations, DAS can provide a continuous acoustic/vibration profile along the entire length of the cable. This makes it ideal for applications where it's important to monitor acoustic/vibration changes over a large area or distance.

Distributed acoustic sensing/distributed vibration sensing (DAS/DVS), also sometimes known as just distributed acoustic sensing (DAS), is a technology that uses optical fibers as widespread vibration and acoustic wave detectors. Like distributed temperature sensing (DTS), DAS/DVS allows continuous monitoring over long distances, but instead of measuring temperature, it measures vibrations and sounds along the fiber.

DAS/DVS operates as follows. Light pulses are sent through the fiber optic sensor cable. As the light travels through the cable, vibrations and sounds cause the fiber to stretch and contract slightly. These tiny changes in the fiber's length affect how the light interacts with the material, causing a shift in the backscattered light's frequency. By analyzing the frequency shift of the backscattered light, the DAS/DVS system can determine the location and intensity of the vibrations or sounds along the fiber optic cable.

DAS/DVS offers several advantages over traditional point-based vibration sensors: High spatial resolution: It can measure vibrations with high granularity, pinpointing the exact location of the source along the cable; Long distances: It can monitor vibrations over large areas, covering several kilometers with a single fiber optic sensor cable; Continuous monitoring: It provides a continuous picture of vibration activity, allowing for better detection of anomalies and trends; Immune to electromagnetic interference (EMI): Fiber optic cables are not affected by electrical noise, making them suitable for use in environments with strong electromagnetic fields.

DAS/DVS technologies have proven useful in a wide range of applications, including: Structural health monitoring: Monitoring bridges, buildings, and other structures for damage or safety concerns; Pipeline monitoring: Detecting leaks, blockages, and other anomalies in pipelines for oil, gas, and other fluids; Perimeter security: Detecting intrusions and other activities along fences, pipelines, or other borders; Geophysics: Studying seismic activity, landslides, and other geological phenomena; and Machine health monitoring: Monitoring the health of machinery by detecting abnormal vibrations indicative of potential problems.

Distributed Fiber Optic Sensing (DFOS) technology leverages the existing fiber infrastructures as a potential sensing media, enabling a wide-range, real-time, and continuous monitoring of surrounding environment perception without the need to introduce additional sensing devices. DFOS has been successfully employed in diverse applications including road traffic monitoring, intrusion detection, earthquake detection, pipeline leakage monitoring and structure change detection.

Operational telecommunications optical fiber cable networks hold substantial potential for environmental perception and sensing applications. DFOS technology transforms existing communication cables into individual sensors distributed at every meter along the optical fiber cable, with all the measurements being synchronized. As a result, this sensing technology can be employed to detect events related to both infrastructure itself and its surrounding environments.

As previously noted, a basic principle behind the DFOS is that optical fiber cable conditions such as a change of strain or temperature on the optical fiber cable can influence the properties of the light signal traveling through an optical fiber. When pulsed light is launched into an optical fiber sensing cable, a small fraction of light is backscattered, and its properties are influenced by the fiber cable condition. The backscattered light includes three types of scattering: Raman scattering, Brillouin scattering, and Rayleigh scattering. This methodology gauges alterations in Rayleigh scattering intensity via interferometric phase beating. With coherent detection, the DFOS system retrieves comprehensive polarization and phase information from the backscattering signals, enabling impressive meter-level fiber cable sensor resolution.

Distributed Temperature Sensing (DTS) is a technology that utilizes optical fibers as linear sensors to measure temperature continuously along their length. Instead of using discrete temperature sensors at specific points, a DTS system provides a temperature profile over the entire fiber, which can extend for many kilometers.

DTS technology primarily relies on the interaction of light with the glass structure of the optical fiber, specifically a phenomenon called Raman scattering. When a short pulse of laser light is sent into the fiber, a small portion of the light is scattered back in different wavelengths. This backscattered light contains information about the temperature at the point of scattering.

As previously noted, there are three main types of scattering, but DTS systems mainly analyze Raman scattering:

Rayleigh Scattering: Light scattered back at the same wavelength as the incident light. This is used in Optical Time Domain Reflectometry (OTDR) for locating faults in fibers but not primarily for temperature sensing in DTS.

Brillouin Scattering: Light scattered back with a slight frequency shift that is sensitive to both temperature and strain. Some DTS systems use Brillouin scattering, but careful design is needed to differentiate between temperature and strain effects.

Raman Scattering: Light scattered back at different wavelengths (Stokes and anti-Stokes lines). The intensity ratio between the anti-Stokes and Stokes lines is directly dependent on the temperature at the scattering point. The anti-Stokes line is more temperature sensitive.

Operationally, temperature and location are determined as follows.

Laser Pulse: A DTS system sends short pulses of laser light into one end of an optical sensor fiber.

Backscattering: As the light pulse travels along the fiber, Raman scattering occurs continuously at every point. This generates backscattered light with Stokes and anti-Stokes components.

Detection and Analysis: The DTS instrument at the same end of the fiber detects the returning backscattered light.

Temperature Calculation: The instrument measures the intensity of the Stokes and anti-Stokes lines. The ratio of these intensities is used to calculate the temperature at the point where the scattering occurred.

Location Determination: The location of the temperature measurement along the fiber is determined by measuring the time it takes for the backscattered light to return. This is similar to how radar works; the longer the return time, the farther the scattering point is from the instrument. This technique is known as Optical Time Domain Reflectometry (OTDR).

As we have noted, key features and advantages of DFOS and DTS in particular include at least the following.

Continuous Monitoring: Provides a temperature profile along the entire length of the fiber, offering much more information than discrete sensors.

Long Distances: Can monitor temperatures over distances of many kilometers (up to 100 km or more with some systems).

High Spatial Resolution: Can achieve temperature measurements with a spatial resolution down to one meter or even better in some specialized systems.

Immunity to Electromagnetic Interference (EMI): Optical fibers are immune to EMI, making DTS suitable for industrial environments with electrical noise.

Safety in Hazardous Environments: Low laser power levels used in many DTS systems make them safe for use in potentially explosive atmospheres.

Cost-Effective for Large Areas/Distances: Reduces the need for numerous individual sensors and their associated wiring and installation costs.

Versatile Applications: Used in a wide range of industries for various monitoring tasks.

Accordingly, and as will be readily understood and appreciated by those skilled in the art, distributed temperature sensing is a powerful technology that leverages the properties of optical fibers and light scattering to provide continuous and spatially resolved temperature measurements over long distances, offering significant advantages for a wide array of monitoring applications.

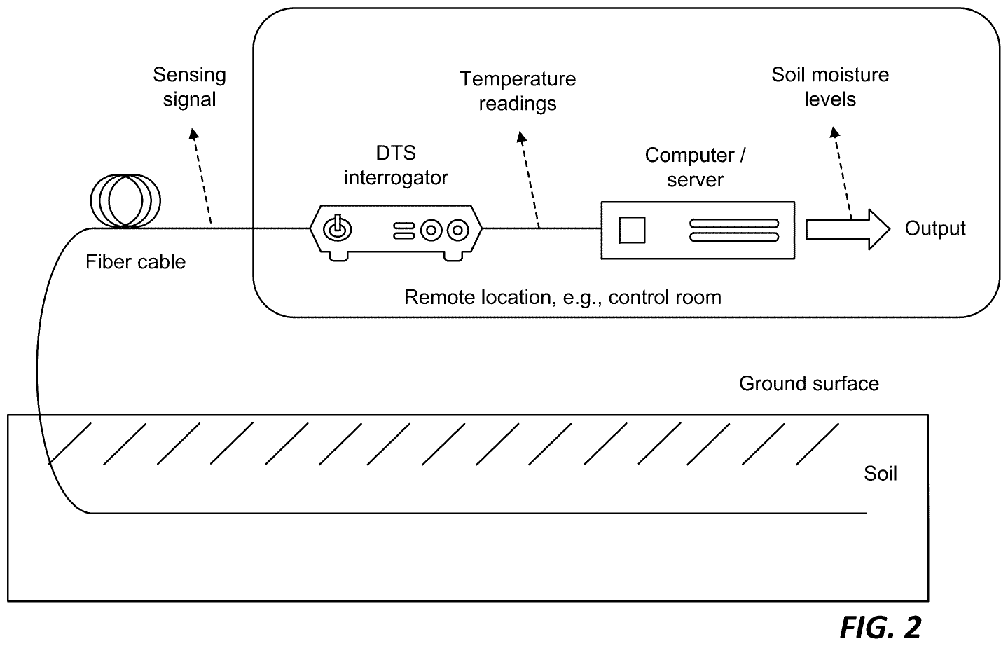

FIG. 2 is a schematic diagram showing operational details of an illustrative system according to aspects of the present disclosure.

With reference to that figure, those skilled in the art will understand and appreciate that a distributed temperature sensing (DTS) interrogator reads and records soil temperature at meter-level spatial resolution continuously from a fiber optic cable buried—or otherwise in substantial contact with—target soil. Once that DTS data is collected, a computer or server, by comparing the temperature changing speed across hours or a day between soil (from the DTS measurements) and ambient air temperatures (from weather report or weather station readings), soil heat capacity can be calculated. Since the soil heat capacity is related to its moisture content, the soil moisture levels are estimated by comparing the calculated soil heat capacity with the heat capacity of dry soil or calculated by heat capacity models of soil-water mixtures. The implementation schematic is shown in the figure.

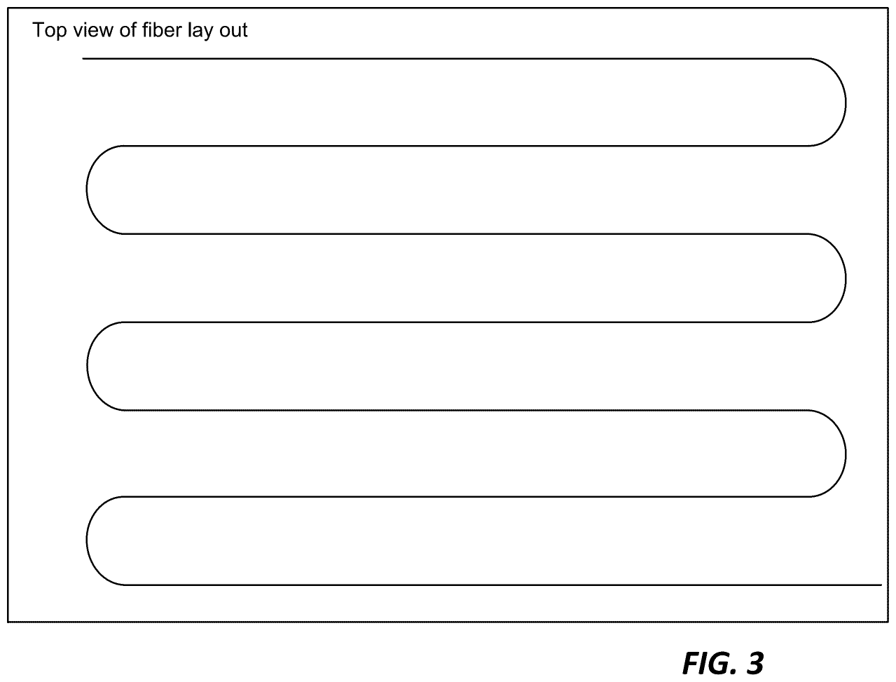

FIG. 3 is a schematic diagram showing an illustrative fiber optic sensor layout over a large geographic area in which DTS measurements are made according to aspects of the present disclosure.

As illustratively shown in this figure, the fiber optic sensor is laid throughout a sensor area in a serpentine manner such that a large area is DTS monitored.

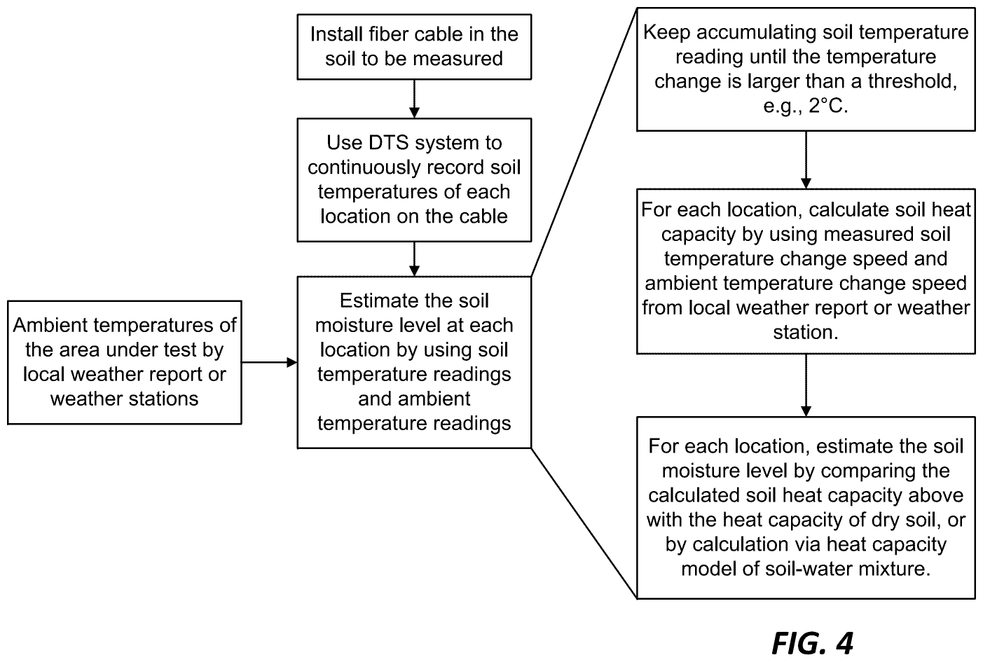

FIG. 4 is a schematic flow diagram showing illustrative steps involved in DTS and soil moisture measurements and determinations according to aspects of the present disclosure.

A fiber optic sensor cable in substantially buried in a soil area of interest. the soil. The cable's location and depth should be as close as possible to the target positions where moisture levels need be measured. Specific layout patterns can be used to cover a large area by one single fiber cable, for example as shown in figure FIG. 3.

DTS is operated and provides continuous reading of soil temperature from every single location point along the length of the fiber optic sensor cable at a meter-level spatial resolution.

At each single location point along the length of the fiber optic sensor cable, by accumulating multiple hours of soil temperature readings, the temperature changing speed during this time is calculated. Meanwhile, ambient temperature changing speed is calculated using ambient temperature readings obtained from local weather reports or a weather station. Then the soil heat capacity is derived by comparing the soil temperature changing speed and the ambient air temperature changing speed.

At each single location point on the fiber cable, the soil moisture level is estimated by comparing the calculated soil heat capacity above with the heat capacity of dry soil. Alternatively, the soil moisture level can be calculated by heat capacity model of soil-water mixtures.

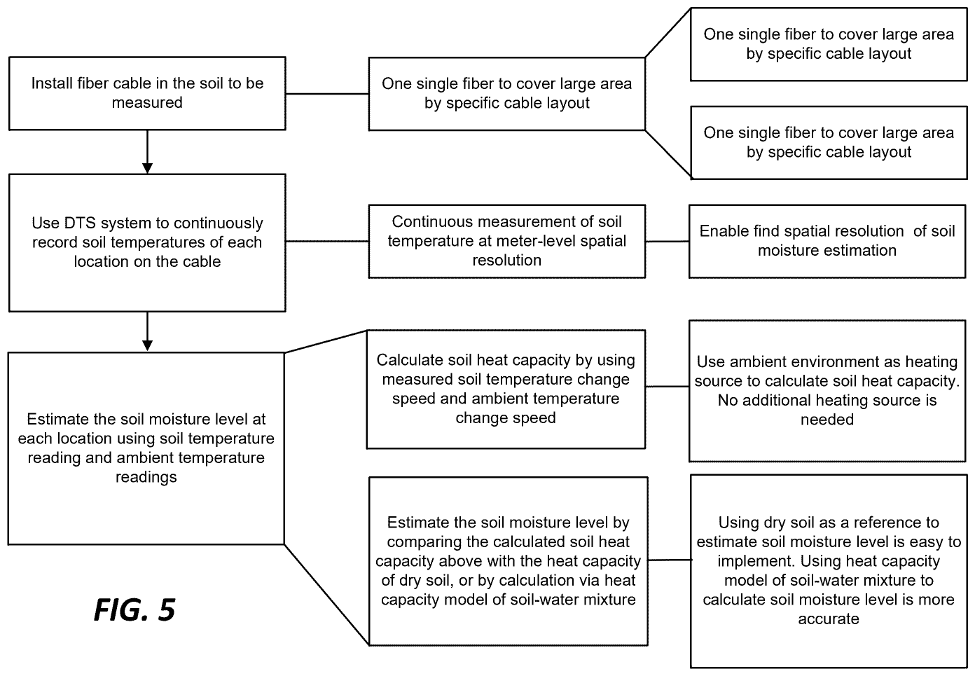

FIG. 5 is a schematic flow diagram showing illustrative steps involved in DTS soil moisture measurements and determinations according to aspects of the present disclosure.

From this figure, a slightly more detailed step by step operation may be described as follows.

Bury a fiber optic sensor cable in the soil. The cable's location and depth should ideally be as close as possible to any target positions where moisture levels need to be measured. Specific layout patterns can be used to cover a large area by one single fiber optic sensor cable.

Connect and operate a DTS interrogator and analysis system to one end of the fiber. The DTS interrogator records continuous reading of soil temperature from every single location point along the fiber optic sensor cable at a meter-level spatial resolution.

At each single location point along the fiber optic sensor cable, accumulate multiple soil temperature readings, until the soil temperature changes are larger than a threshold, e.g., 2° C. Meanwhile, keep recording the ambient temperature readings of the area under test from local weather reports or weather stations.

The temperature changing speeds of both soil and ambient air during this time period is then calculated. From these changing speed determinations, the soil heat capacity is derived by comparing the soil temperature changing speed and the ambient air temperature changing speed.

The soil moisture level is then estimated by comparing the calculated soil heat capacity determined above with the heat capacity of dry soil. Alternatively, the soil moisture level can also be calculated by heat capacity model of a soil-water mixture.

The above steps to estimate the soil moisture level can be done for each location along the length of the fiber optic sensor cable in parallel.

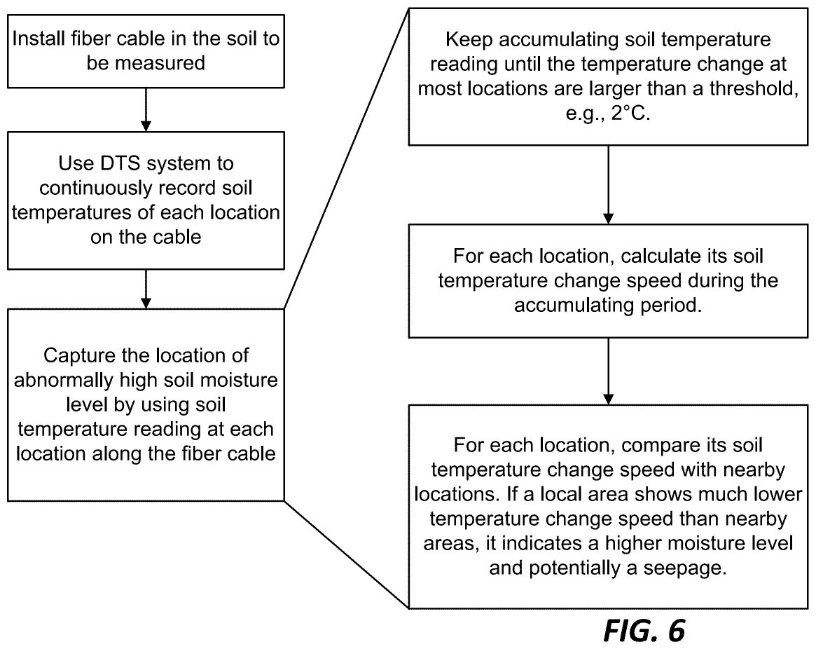

FIG. 6 is a schematic flow diagram showing illustrative steps involved in DTS soil temperature measurement and soil moisture levels and determining a seepage according to aspects of the present invention.

With reference to this figure, it may be observed that much of the technique for soil moisture can be applied to water-seepage determinations in the same area under evaluation for water moisture.

In this inventive manner, a water seepage detection system and method by DTS is implemented for water seepage detection in—for example—earthen embankment dams. Since this application measures soil moisture level at a meter-level of spatial resolution, it can be used to pinpoint water seepage at an early stage by detecting an abnormally high soil moisture level as compared to surrounding locations without seepage.

Advantageously, the hardware employed in our inventive water seepage systems and methods are substantially the same as those described previously for soil moisture level detection. Differences in processing are noted as:

Capturing the location of abnormally high soil moisture levels by using soil temperature readings at each location along the fiber cable, and for each location, compare its soil temperature change speed with nearby locations. If a local area shows much lower temperature change speed than nearby areas, it indicates a higher moisture level and a possible seepage.

As those skilled in the art will understand and appreciate, our inventive systems and methods for water seepage determination offer the following surprising advantages.

As compared with traditional point sensors for seepage detection, our inventive systems and methods use only one fiber optic sensor cable to monitor a large area without the requirement of power supply and communication facility in the field, which greatly reduce the number of sensors and maintenance cost.

As compared with Fiber Bragg Grating (FBG) based fiber optic sensing approaches, our inventive systems and methods provides many monitoring locations, which results in longer monitoring distance or fiber spatial resolution.

Finally, as compared with actively heated fiber sensing approaches, our inventive systems and methods use environmental temperature variation as heat source to eliminate the requirement of a field heating element, which greatly reduces system complexity and cost.

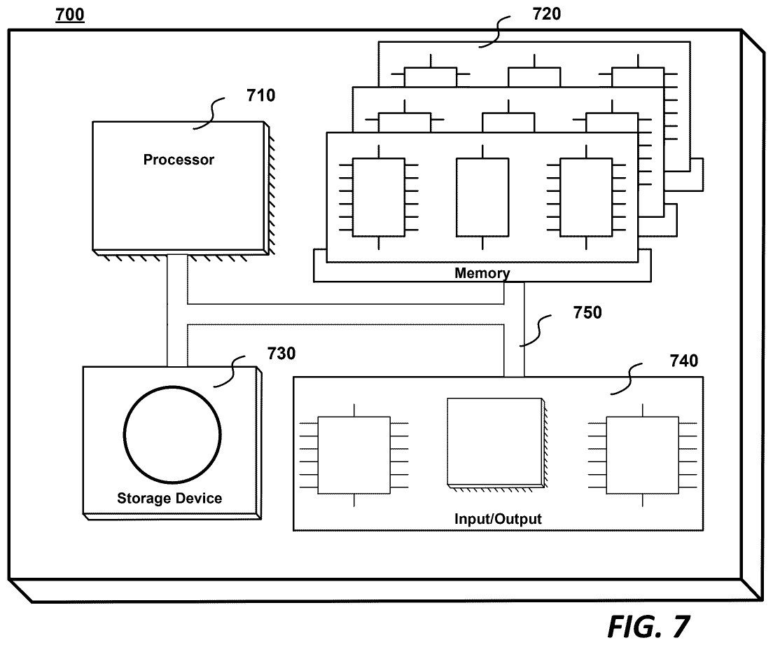

FIG. 7 is a schematic block diagram of an illustrative computing system that may be programmed with instructions that when executed produce the methods/algorithms according to aspects of the present invention.

As may be immediately appreciated, such a computer system may be integrated into another system such as a router and may be implemented via discrete elements or one or more integrated components. The computer system may comprise, for example, a computer running any of a number of operating systems. The above-described methods of the present disclosure may be implemented on the computer system 700 as stored program control instructions.

Computer system 700 includes processor 710, memory 720, storage device 730, and input/output structure 740. One or more input/output devices may include a display 745. One or more busses 750 typically interconnect the components, 710, 720, 730, and 740. Processor 710 may be a single or multi core. Additionally, the system may include accelerators etc., further comprising the system on a chip.

Processor 710 executes instructions in which embodiments of the present disclosure may comprise steps described in one or more of the Drawing figures. Such instructions may be stored in memory 720 or storage device 730. Data and/or information may be received and output using one or more input/output devices.

Memory 720 may store data and may be a computer-readable medium, such as volatile or non-volatile memory. Storage device 730 may provide storage for system 700 including for example, the previously described methods. In various aspects, storage device 730 may be a flash memory device, a disk drive, an optical disk device, or a tape device employing magnetic, optical, or other recording technologies.

Input/output structures 740 may provide input/output operations for system 700.

At this point, those skilled in the art will understand and appreciate that we introduce a Deep Phase-Magnitude Network (DFMN) and point out that combining the filtering in time domain and frequency domain can significantly enhance the classification accuracy and improve the domain generalization ability. We divide the raw fiber sensing data into magnitude response and phase response for parallel feature representation learning. Furthermore, we propose a Phase Frequency Learnable Filter (PFLF) specifically designed for phase component learning, which effectively determines the frequency components crucial for enhancing rain detection accuracy. In the end, we formulate the phase-magnitude channel within a dual-path network and subsequently fuse the features for a comprehensive analysis. Extensive experiments and ablation studies demonstrate the effectiveness of our proposed method.

While we have presented our inventive concepts and description using specific examples, our invention is not so limited. Accordingly, the scope of our invention should be considered in view of the following claims.

1. A computer-implemented method for monitoring soil moisture using distributed temperature sensing (DTS), the method comprising:
collecting a plurality of soil temperatures using DTS;
determining heat capacities of the soil;
determining soil moisture levels from the determined heat capacities of the soil.

2. The method of claim 1 further comprising determining ambient temperatures of an aera in which the DTS temperatures are collected.

3. The method of claim 2 further comprising determining, using the collected temperatures of the soil and the determined ambient temperatures, soil temperature changing speeds.

4. The method of claim 3 further comprising determining the soil moisture level by comparing the determined heat capacities of the soil with heat capacities of dry soil.

5. The method of claim 4 wherein the plurality of collected soil temperatures are made at meter-level spatial resolution.

6. The method of claim 5 wherein the ambient temperatures of the area are determined from local weather reports and/or weather stations.

7. The method of claim 3 further comprising determining the soil moisture level from a heat capacity model of a soil-water mixture.
